(19) United States
(12) Patent Application Publication
(10) Pub. No.: US 2023/0172202 A1
(43) Pub. Date: Jun. 8, 2023

(54) INSECT AND NON-INSECT ARTHROPOD PEST REPELLENT COMPOSITIONS AND METHODS

(71) Applicant: Livful Inc., Temple, TX (US)

(72) Inventors: Thomas P. DOOLEY, Pinson, AL (US); Eduard Casas-Terradellas, Manchester (GB); Tycho Speaker, Santa Cruz, CA (US)

(21) Appl. No.: 18/059,475

(22) Filed: Nov. 29, 2022

Related U.S. Application Data
(60) Provisional application No. 63/285,145, filed on Dec. 2, 2021.

(51) Int. Cl. A01N 37/20; A01N 25/28; A01P 17/00

(52) U.S. Cl. CPC A01N 37/20; A01N 25/28; A01P 17/00

(57) ABSTRACT

The present invention relates to a topical consumer product which is an insect or non-insect arthropod pest repellent microencapsulated emulsion containing non-DEET insect or non-insect arthropod pest repellent active ingredients, such as IR3535®. The insect or non-insect arthropod pest repellent microencapsulated emulsion exhibits long-duration insect or non-insect arthropod pest repellency efficacy over conventional pest repellant compositions. Such a topical consumer product exhibits performance characteristics which include product stability with respect to the physical and chemical properties of the formulation and chemical properties of the active ingredient, and provides the required shelf-life stability and long-duration insect repellency desired for a commercial insect or non-insect arthropod pest repellent product.

### BACKGROUND OF THE INVENTION

This invention relates generally to compositions and methods of sustained release encapsulated emulsions as topical lotion formulations with long-duration repellency efficacy, containing non-DEET (N,N-diethyl-m-toluamide) insect or non-insect arthropod pest repellent active ingredients, such as IR3535® (ethyl butylacetylaminopropionate; Merck KGaA), and having improved chemical stability characteristics, namely pH stability and active ingredient stability.

Microencapsulation of a topical insect or non-insect arthropod pest repellent formulation can decrease the transdermal penetration of a common active ingredient DEET (N,N-diethyl-m-toluamide) (1, 2). Furthermore, microencapsulation can prolong evaporation rates of the active ingredient, thus providing a sustained release mode of action (2).

U.S. Pat. No. 8,039,015 discloses methods and compositions for microencapsulation of chemicals using Lewis Acid—Lewis Base bonding interactions, including insect or non-insect arthropod pest repellent active ingredients, and including DEET. U.S. Pat. No. 8,039,015 discloses microcapsules containing a water-immiscible core material surrounded by a wall-forming Lewis acid (e.g., carboxymethylcellulose) and a wall-forming Lewis base (e.g., benzalkonium chloride). U.S. Pat. No. 8,039,015 specifically discloses that the resulting sodium carboxymethylcellulose plus benzalkonium chloride capsules of an aqueous suspension of microencapsulated DEET are shelf-stable, showing no evidence of Ostwald ripening or coalescence after months of storage, but some stratification (i.e., separation) of the suspension is observable after more than one month. These patents did not disclose long-term stability consistent with commercial grade shelf life or insect repellency efficacy of the encapsulated microemulsions.

PCT International Application Publication No. WO 2020/065541 and US Application Publication No. 2022/0047472 disclose methods of preparing translucent encapsulated microemulsions with a Lewis acid and a Lewis base. The translucent microemulsions may contain alginic acid (algin), chitosan, xanthan gum, and an insect repellent, such as ethyl butylacetylaminopropionate (IR3535®). PCT International Application Publication No. WO 2020/065541 and US Application Publication No. 2022/0047472 are silent with regard to chemical stability of pH (e.g., reduction in pH) and the active ingredient of the encapsulated microemulsion formulation. The disclosure is silent with regard to compositions comprising any IR3535®-containing encapsulated microemulsion, or achieving a chemically-stable repellent formulation containing IR3535, or any evidence of insect repellency efficacy of the encapsulated microemulsions.

PCT International Application Publication No. WO 20209908, PCT International Application Publication No. WO 20209907, and European Application Publication No. EP3897522 disclose sustained release microcapsules, which may contain chitosan, alginate, xanthan gum, citric acid for pH adjustment, and an insect repellent, but did not disclose IR3535®.

European Application Publication No. EP 2898774, European Application Publication No. EP 1464385, European Application Publication No. EP 1247568, and U.S. Pat. No. 6,534,091 disclose microcapsules, which may contain ionic polymers, sodium alginate, xantham gum, chitosan, citric acid for pH adjustment, and an insect repellent, such as IR3535®.

U.S. Pat. No. 9,730,867 discloses compositions comprising microcapsules, which may contain a Lewis acid and Lewis base, chitosan, citric acid, sodium citrate, and an insect repellent, but did not disclose IR3535e.

European Application Publication No. EP 1479432 discloses microcapsules, which may contain polymers, such as chitosan, pH regulators, buffer mixtures and an insect repellent, such as IR3535®.

Significantly, each of the preceding disclosures are silent with respect to a composition comprising microencapsulated emulsions of an insect repellant, such as IR3535®, wherein the composition exhibits stability of the active agent, stability of pH, and long-duration repellency efficacy (e.g., 12, or more hours on human skin). These highly desirable features of a composition may facilitate the required shelf-life stability such that the composition may be acceptable to a governmental regulatory agency and/or sold commercially as an insect or non-insect arthropod pest repellent product.

The combination of essential features of the present invention: (a) encapsulation of IR3535® within an emulsion using Lewis Acid—Lewis Base adducts; (b) demonstrated evidence of long-term pH stability of the formulation using sodium citrate—citric acid buffer; (c) demonstrated evidence of long-term chemical stability of IR3535® using sodium citrate—citric acid buffer; and (d) demonstrated evidence of long-duration mosquito repellency by IR3535®; and within the context of a cosmetically-appealing lotion formulation, provide the required shelf-life stability and long-duration insect repellency desired for a commercial insect or non-insect arthropod pest repellent product.

IR3535® (ethyl butylacetylaminopropionate) has been widely disclosed for greater than two decades to be an effective and safe mosquito repellent active ingredient. This active ingredient has been included in multiple commercial consumer products in various countries.

The efficacy of IR3535®-containing topical formulations (e.g., sprays and lotions) against mosquitos have been ranked relative to DEET-containing formulations (3-10). Comparisons of the two active ingredients have also been made using sandflies—the vectors of Leishmaniasis (11) and the insect vectors of trypanosomiasis—Chagas disease (12, 13). In general, in repellency efficacy comparisons of the same or similar concentrations of active ingredients, IR3535® is less effective or comparable to DEET. For example, the mean complete protection time (mean CPT) for human subjects in a field trial in the Everglades of Florida for 25% IR3535® (in ethanol) was 3.0 hours versus 5.6 hours for 25% DEET (4). The CPT is the number of minutes (or hours) elapsed between topical application and the first landing and/or probing (biting) of the biting insect. The CPT may alternatively be based upon the time of the confirmed first bite (CFB), which represents the first subject within a group of subjects experiencing his/her second bite or confirmatory bite. Although these publications disclose the duration of repellency efficacy for compositions containing IR3535®, they do not present comparisons of standard formulations (e.g., in ethanol or an emulsion) versus sustained release formulations that are intended to enhance long-duration repellency efficacy.

Although the potency of DEET can be greater than that of IR3535®, the latter has several distinct advantages over DEET for use on human or nonhuman mammalian skin. Most notably IR3535® lacks an odor and produces a non-oily/non-greasy feel for preferred cosmetic appeal to consumers.

Although the references cited herein may disclose microcapsules and/or emulsions or microemulsions comprising an insect repellent, such as IR3535®, the cited references do not disclose compositions or formulations which provide for a shelf-stable commercial microencapsulated emulsion, for example a lotion product, for the long-duration efficacious topical repellency of insect and non-insect arthropod pests.

The prolonged efficacy of IR3535® repellents against mosquitoes and blacklegged ticks in North America has been studied and reported in Carroll, J P (15). Controlled-release formulations of IR3535®, at 10% in lotion and at 20% in pump spray and aerosol, against mosquitoes in the field and blacklegged ticks were evaluated in a laboratory setting. Complete protection times ranged from 7.1 to 10,3 h for mosquitoes and from 9.1 to 12.2 h for blacklegged ticks.

Field evaluation of the efficacy of proprietary repellent formulations with IR3535® and Picaridin against Aedes aegypti has been reported in Naucke TJ (16). Seven proprietary repellent formulations (3 hydro-alcoholic spray solutions and 4 skin lotions) with active ingredient IR3535® (ethyl butylacetylaminopropionate) or Picaridin (hydroxyethyl isobutyl piperidine carboxylate) were tested in a field study on 10 test persons over a period of 10 h for their efficacy at preventing bites. The tests were conducted on field populations of the yellow fever mosquito Aedes aegypti. The concentration of the active substances ranged from 10% to 20%. All the tested samples provided lasting protection (time to first bite) over several hours: ranging from 5 h 20 min to 6 h 50 min with a mean of approximately 6 h. The longest protection until the second bite (-first confirmation bite) was approximately 7 h 40 min, whereas the shortest protection was 6 h 50 min. The longest protection until the third bite (=second confirmation bite) was 8 h 35 min.

The instant invention provides a topical repellent formulation containing encapsulated IR3535® with formulation stability and long-duration efficacy against mosquitos and non-insect arthropods (e.g., at least 12 hrs, at least 12.5 hrs, at least 13 hours, including 14, 15, 16 or more hours on human skin). Although numerous commercial products containing IR3535® have been and are marketed, none provide sustained release encapsulated topical formulations with long-duration mosquito and and non-insect arthropod repellency efficacy (e.g., 12.5 or more hours) containing non-DEET insect or non-insect arthropod pest repellent active ingredients, such as IR3535® and having chemical stability characteristics which provide for a shelf-stable commercial product over conventional products.

### BRIEF SUMMARY OF THE INVENTION

In a first aspect, the present invention is a composition of an insect or non-insect arthropod pest repellent comprising one or more active ingredients selected from ethyl butylacetylaminopropionate (IR3535®) and other non-DEET active ingredients, one or more emollients, one or more emulsifiers, one or more Lewis Acids for microencapsulation, one or more Lewis Bases for microencapsulation, one or more viscosity thickeners, one or more film formers, a pH buffer of trisodium citrate or hydrates thereof and citric acid, and water.

In another aspect, the present invention is an insect or non-insect arthropod pest repellent composition consisting essentially of ethyl butylacetylaminopropionate (IR3535®) and one or more emollients, one or more emulsifiers, one or more Lewis Acids for microencapsulation, one or more Lewis Bases for microencapsulation, one or more viscosity thickeners, one or more film formers, a pH buffer of trisodium citrate, or hydrates thereof, and citric acid, and water.

In an embodiment of the insect or non-insect arthropod pest repellent composition of the invention, the emollient is isoeicosane, wherein the emulsifiers are selected from sorbitan oleate, polysorbate 20, and combinations thereof, wherein the Lewis Acids for microencapsulation are selected from algin, xanthan gum, and combinations thereof, wherein the Lewis Base for microencapsulation is chitosan, wherein the viscosity thickeners are selected from polyamide-3, cetyl alcohol, carrageenan, and combinations thereof, and wherein the film formers are selected from dimethicone, acrylates copolymer, and combinations thereof.

In a further embodiment, the insect or non-insect arthropod pest repellent composition comprises ethyl butylacetylaminopropionate (IR3535®) which is present in the composition at a concentration of approximately 2 to 22 (wt) percent.

In a further embodiment of the insect or non-insect arthropod pest repellent composition, the Lewis Acids and Lewis Bases for encapsulation in aggregate do not exceed 1 (wt) percent.

In a further embodiment of the insect or non-insect arthropod pest repellent composition, the pH is approximately 5.0 to 6.5, and the pH buffer is approximately 0.5 to 3 (wt) percent.

In an embodiment of the insect or non-insect arthropod pest repellent composition, the composition is in the form of a microencapsulated emulsion.

In an embodiment of the insect or non-insect arthropod pest repellent composition, the composition is in the form of a concentrated microencapsulated emulsion, wherein the concentrated microencapsulated emulsion may be diluted with an aqueous solution immediately prior to administration.

In an embodiment of the insect or non-insect arthropod pest repellent composition optionally comprises a fragrance.

In an embodiment of the insect or non-insect arthropod pest repellent composition, the composition exhibits an imputed or formal shelf life of at least 12 months.

In a further aspect, the invention pertains to a method of treating skin, hair or clothing of a mammal to repel insects or non-insect arthropod pests comprising applying the insect or non-insect arthropod pest repellent composition to the skin, hair and/or clothing of the mammal.

In an embodiment, the mammal is a human, horse, cow, dog, cat, or other domesticated mammal.

In an embodiment, the insects are selected from one or more species of mosquito, fly, sandfly, black fly, louse, chigger, bed bug, a vector of trypanosomiasis, bee, Halictid, wasp, and hornet, and wherein the non-insect arthropod pests are selected from one or more species of tick, chigger (Trombiculidae), spider, and scorpion.

In a further embodiment, a single application of the insect or non-insect arthropod pest repellent composition to the skin, hair, or clothing repels insects of one or more species for a minimum of 10 hours, a minimum of 12 hours, a minimum of 12.5 hours, a minimum of 13 hours, or a minimum of 14 hours.

In an embodiment, the application of an insect or non-insect arthropod pest repellent composition further prevents symptoms of insect-borne diseases, and wherein the diseases include one or more of malaria, dengue fever, Lyme disease (Borreliosis), Leishmaniasis, Chagas disease (trypanosomiasis), Zika fever, equine encephalitis, and yellow fever.

In another aspect, the present invention is a composition of an insect or non-insect arthropod pest repellent consisting essentially of ethyl butylacetylaminopropionate (IR3535®) as the active ingredient at a concentration of approximately 2 to 22 percent, one or more emollients, one or more emulsifiers, one or more Lewis Acids for microencapsulation, one or more Lewis Bases for microencapsulation, one or more viscosity thickeners, one or more film formers, a pH buffer of trisodium citrate, or hydrates thereof, and citric acid, and water.

In a third aspect, the present invention is a method of treating skin, hair, or clothing of a mammal to repel insects or non-insect arthropod pests by the application of an insect repellent composition or non-insect arthropod pest composition consisting essentially of one or more active ingredients selected from ethyl butylacetylaminopropionate (IR3535®) and other non-DEET active ingredients, one or more emollients, one or more emulsifiers, one or more Lewis Acids for microencapsulation, one or more Lewis Bases for microencapsulation, one or more viscosity thickeners, one or more film formers, trisodium citrate or hydrates thereof and citric acid for pH buffering, and water.

In an embodiment, the non-DEET active ingredients, include natural active ingredients, for example, oil of lemon eucalyptus (OLE) or clove leaf oil, at concentrations of 10 and 20% w/w, and 5% w/w respectively.

### DETAILED DESCRIPTION OF THE INVENTION

Before describing preferred embodiments of the present invention in detail, definitions important for understanding the meaning and scope of the present invention are given.

CFB means confirmed first bite.

CPT means complete protection time.

DEET means N,N-diethyl-m-toluamide.

EPA means US Environmental Protection Agency.

FDA means US Food and Drug Administration.

GMP means Good Manufacturing Practice.

INCI means International Nomenclature Cosmetic Ingredient.

The term IR3535® means ethyl butylacetylaminopropionate, also referred to as ethyl-N-acetyl-N-butyl-beta alaninate.

OTC means over-the-counter.

The term “pH” means the log of the hydronium (H₃O+) or proton concentration.

The term “RT” means ambient room temperature, and often considered as ca. 20°-25° C.

US means United States of America.

v/v means volume per volume.

w/v means weight per volume.

w/w means weight per weight.

An “emulsifier” means a substance that stabilizes an emulsion.

The term “microcapsules” means encapsulated microscopic particles.

Emulsions are liquid mixtures of immiscible hydrophobic chemicals (e.g., oils) and hydrophilic chemicals (e.g., water). To overcome the immiscibility of the oil and water phases, one or more emulsifiers and/or surfactants may be included. Emulsions are also referred to as “macroemulsions” and are optically opaque.

As used herein, the term “surfactant” refers to a substance which lowers the surface tension of the medium in which it is dissolved, and/or the interfacial tension with other phases. Accordingly, the surfactant is positively adsorbed at the liquid/vapor or at other interfaces. Non-limiting, exemplary surfactants suitable for use in the disclosed compositions and/or microemulsions include those surfactants described in McCutcheon's Emulsifiers and Detergents (North American and International Editions, by Schwartz, Perry and Berch) and a variety of other surfactant references commonly known to surfactant formulators.

As used herein, the term “microemulsion” refers to an emulsion that is optically clear (i.e., translucent) and thermodynamically stable. Microemulsions have droplet sizes smaller than those of emulsions.

The microencapsulated emulsions of the present disclosure may be oil-in-water (o/w) or water-in-oil (w/o) and are not limited to a particular dispersion phase structure. For example, emulsions of the present disclosure may comprise droplets, microdroplets, and/or bicontinuous microstructure.

Microencapsulation means a process in which tiny particles or droplets are surrounded by a polymeric coating to yield a shell, thus yielding encapsulated particles or encapsulated droplets.

“Encapsulated emulsions” or “encapsulated microemulsions” mean tiny particles in a liquid mixture consisting of an internal emulsion or microemulsion, wherein the active ingredient is stored, and surrounded by an outermost polymeric encapsulation wall, layer, or shell, wherein the latter provides structural support.

As used herein, the term “Lewis acid” is a molecular entity that is an electron-pair acceptor and therefore able to react with a Lewis base to form a Lewis adduct, by sharing the electron pair furnished by the Lewis base. In some embodiments, a Lewis acid is an amphiphilic cationic species.

As used herein, a “Lewis base” is a molecular entity able to provide a pair of electrons and thus capable of coordination with a Lewis acid, thereby producing a Lewis adduct. In some embodiments, a Lewis base is an amphiphilic anionic polymer. Non-limiting, exemplary Lewis acids and Lewis bases suitable for use in the disclosed microemulsions are provided herein. Unless otherwise specified, or clear from the context, mention of the presence of a particular Lewis acid or a particular Lewis base as a component of a composition will be understood to encompass either the free acid or base, or the salts of these.

“Shelf life” or “shelf stable” means the length of time for which an item remains usable, fit for the intended use, and/or saleable. With regard to consumer product topical formulations shelf life means the time interval from manufacturing and/or packaging, typically in months or years, that a formulation composition is anticipated to meet performance testing standards regarding chemical and/or physical properties. Failure to meet the performance testing standards within the appropriate time interval serve as evidence(s) of insufficient stability. A stable repellant composition with a “formal” and/or “imputed” shelf life of at least 12 months, and preferably 18 months, more preferably 24 months, and even more preferably 36 months at ambient room temperature (20°-25° C.) is desired for a topical consumer product. The minimal shelf life in months or years may (also) be a specified term of acceptance for product registration by a governmental regulatory agency.

Specific embodiments disclosed herein may be further limited in the claims using “consisting of” or “consisting essentially of language”. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

As used herein, the term “comprising” or “comprises” is intended to mean that the compositions and methods include the recited elements, but not excluding others.

As used herein, the terms “optional” or “optionally” mean that the subsequently described event or condition may need not occur, and that the description includes instances where the event or condition occurs and instances in which it does not.

Insects which are repelled by application of the formulations of the invention are selected from one or more species of mosquito, fly, sandfly, black fly, louse, chigger, bed bug, a vector of trypanosomiasis, bee, Halictid, wasp, and hornet.

Non-insect arthropod pests which are repelled by application of the formulations of the invention are selected from one or more species of tick, chigger (Trombiculidae), spider, and scorpion.

The present invention relates to a topical consumer product (e.g., insect repellent lotion). Such topical consumer products must exhibit various performance characteristics, which performance characteristics include product stability with respect to the physical and chemical properties of the formulation and chemical properties of the active ingredient. A stable composition with a “formal” and/or “imputed” shelf life of more than 10 months, and preferably 12 months, and more preferably 18 months, and more preferably 24 months, and even more preferably 36 months at ambient room temperature (20°-25° C.) is desired for a topical consumer product. Relevant to the shelf life are multiple parameters, such as: (a) physical aspects (e.g., viscosity, texture, color, uniformity, mono-dispersion, non-separation of phases, etc.); (b) overall chemical stability of the formulation, an example of which is pH stability, preferably exhibiting no more than 1.0 pH unit variance (decrease or increase) during the imputed or formal shelf life; (c) the minimal shelf life in months or years that is a specified term of acceptance for product registration by a governmental regulatory agency in a country or region wherein the product is intended to be registered for sale; and (d) chemical stability of the active ingredient, preferably a variance of no more than 5-6% loss of the active ingredient during the imputed or formal shelf life; In other words, if one prepares an initial nominal active ingredient concentration of 20% w/w, then the loss over time must not exceed 0.05×20% w/w or 0.06×20% w/w. Thus, a loss greater than 1.0% or 1.2% of the nominal concentration of the product label is unacceptable (i.e., a concentration below 19.0% or 18.8% w/w respectively).

Whenever sufficient time does not permit the formal establishment of a shelf life, extrapolations of linear regression slopes obtained over shorter time periods at accelerated temperatures, such as 40° C. and/or 50° C., can be used as surrogates to establish an “imputed” shelf life. When comparing ambient room temperature (RT; ca. 20°-25° C.) to 40° C., the shortened time interval's result at elevated temperature is extrapolated or multiplied by a factor based upon empirical results. One may observe that 40° C. accelerates the instability or other failure aspect(s) of the formulation (e.g., reduction in active ingredient, phase separation, flocculation, viscosity changes, etc.) over ambient room temperature by a factor of, for instance, 3×. Thus, in this example if it does not fail at 6 months at 40° C., but fails shortly thereafter, then the imputed ambient room temperature shelf life would be limited to 6 months×3=18 months.

International regulatory agencies state the allowed tolerances (variances) for the content of active ingredient to be maintained during the entire shelf life (imputed or formal) of insect repellents or pesticides. The World Health Organization (WHO) guidelines declare an allowed tolerance of 6% for repellent products with a declared content of active ingredient at 20° C.±2 of 10-25% active ingredient w/w or w/v (Manual on Development and Use of FAO and WHO Specifications for Pesticides). This 6% tolerance is also accepted by the European Chemicals Agency (ECHA), as well as most of the other regulatory authorities. However, the US Environmental Protection Agency (EPA) specifies an active ingredient tolerance of only 5%. Thus, the variance from the nominal label amount or concentration of the active ingredient should not exceed 5 or 6 percent over the designated shelf life of the repellent product. As an example, a product with nominal concentration of 20% w/w of active ingredient should not have a concentration greater than 21% w/w or less than 19% w/w using the US EPA tolerance standard or a slightly higher tolerance in most jurisdictions outside of the US.

Based in part upon the microencapsulation method of U.S. Pat. No. 8,039,015 a lotion formulation containing IR3535® instead of DEET as the repellent active ingredient was prepared. It was tested for product stability parameters at ambient room temperature (ca. 20°-25° C.), as well as at accelerated temperatures of 40° C. and 50° C. This prototype formulation (version 1.0) manifested characteristics of chemical instability, most notably rapid pH loss of 1.0 unit at 16.4 weeks at RT and only 5.1 weeks at 40° C. (Table 2, below), as well as undesired reductions in the concentration of IR3535® at 24 months. These losses in pH and active ingredient have ramifications for imputed and/or formal shelf life and served essentially as the basis upon which the present invention was conceived. Reformulation efforts were required to improve the imputed and/or formal shelf-life characteristics of the prototype repellent product.

These undesirable properties observed during the development of the present invention (i.e., reductions in pH and active ingredient concentration) minimize the imputed and/or formal shelf life of the lotion formulation. The shelf life of an insect repellent product may be approximately 12, 18, 24, 30, or 36 months at ambient (or at appropriate storage condition) temperature. It is typical to prefer at least 18 or 24 months or longer for a consumer product. Products with short shelf lives (e.g., less than 12 months) reduce the commercial value proposition, as any unsold product at the end of the shelf life might have little or no value. Shelf lives may be stated, for example, as “best if used by (date)” or as “the recommended shelf life in months or years from the manufacture date”.

The present invention is focused upon pH buffered repellent compositions that manifest stabilized formulation properties at ambient room temperature (ca. 20-25° C.) and/or accelerated temperatures (e.g., 40° C., 50° C.). Furthermore, the formulations manifested long-duration (10 or more hours) repellency against insects, and in particular mosquitos.

Pharmaceutically acceptable and/or cosmetically acceptable excipients and adjuvants (e.g., preservatives, fragrances, antioxidants, buffers, surfactants, lipophilic agents, alcohols, solubilizers, bonding agents, dispersion agents, viscosity agents, and/or pharmaceutically acceptable salts thereof) may be included in any of the compositions.

The phrase “pharmaceutically acceptable”, as used in connection with compositions of the invention, refers to molecular entities and other ingredients of such compositions that are physiologically tolerable and do not typically produce untoward reactions when administered to a mammal (e.g., human). Preferably, as used herein, the term “pharmaceutically acceptable” means approved by a regulatory agency of the Federal or a state government or listed in the U.S. Pharmacopeia (USP), National Formulary (NF), or other generally recognized pharmacopeia for use in mammals, and more particularly in humans. Active ingredients and inactive ingredients of the present invention may be in the form of pharmaceutically acceptable salts. “Pharmaceutically acceptable salts” refers to those salts which possess the biological properties of the parent compound(s) and which are not biologically or otherwise undesirable.

The compositions of the present invention may comprise one or more excipients selected from multiple classes of ingredients. Excipients which may be used include carriers (e.g., water and/or organic solvents), surface active agents (surfactants) or emulsifying agents, thickening (viscosity) agents, binding agents, dispersion and/or suspension agents, buffering agents, solubilizers, colorants, coatings, disintegrating agents, encapsulation agents, lubricants, preservatives and/or antioxidants, isotonic agents, fragrances, and combinations thereof. The selection and use of suitable excipients are taught in Gennaro, ed., Remington: The Science and Practice of Pharmacy, 20th Ed. (Lippincott Williams & Wilkins 2003), the disclosure of which is incorporated herein by reference.

The compositions of the present invention, consisting essentially of a repellent active ingredient plus multiple excipients, may be in the form of lotions, creams, sprays, solutions, suspensions, emulsions, and gels. Lotions may be preferable for the compositions of the present invention.

A variety of mechanical devices may be used for the compositions of the present invention to dispense the formulations, for instance pump applicators, spray applicators, sachets, and compressible tube dispensers. The devices may deliver calibrated unit doses, for instance in the cases of fluid dosage forms.

The compositions of the present invention may be prepared in a pack or dispenser device which may contain one or more unit dosage forms containing the active ingredient(s). The primary packaging may, for example, comprise metal and/or plastic foil. Compositions of the invention formulated in a compatible pharmaceutical and/or cosmetic carrier may also be prepared, placed in an appropriate container.

The compositions of the present invention may include surfactants or emulsifier agents, such as sorbitan oleate, polysorbate 20, phospholipids (e.g., lecithin), and sodium lauryl sulfate, among other choices. Surfactants help solubilize otherwise insoluble active and/or inactive ingredients. Surfactants facilitate water-in-oil and oil-in-water emulsions or suspensions.

The compositions of the present invention may include hydrophobic components, such as lipids, oils (e.g., isoeicosane, isopropyl myristate, and isopropyl palmitate), plant-derived oils, fatty acids, unsaturated fatty acids, waxes, petrolatum, lanolin, etc.

The compositions of the present invention may include pharmaceutically or cosmetically acceptable fluidic carriers (e.g., water, ethanol, glycerol), suspending and emulsifying agents (e.g., sorbitan oleate, polysorbate 20, lecithin, sodium lauryl sulfate), viscosity agents (e.g., polyamide-3, cetyl alcohol, carrageenan, cellulose, methyl cellulose, polyethylene glycols, or Carbomer Homopolymer Type A), film formers (e.g., dimethicone, acrylates copolymer), preservatives (e.g., phenoxyethanol, ethylhexylglycerin, a paraben), fragrances, stabilizing agents such as antioxidants (e.g., butylated hydroxytoluene, butylated hydroxyanisole, citric acid), and the like can be added.

The compositions of the present invention may include one or more Lewis Acids (e.g., algin, also referred to as alginic acid, or xanthan gum) and one or more Lewis Bases for encapsulation of emulsions (e.g., chitosan). Lewis acids and bases may be polymeric. Lewis acid—Lewis base adducts are formed by the bonding between a Lewis acid and a Lewis base, without the simultaneous loss of a leaving group or moiety. See U.S. Pat. No. 8,039,015, US Application Publication No. 2022/0047472, Kasting et al., 2008 (1), and Karr et al., 2012 (2), which may be relevant in the selection of Lewis Acid and Lewis Base chemicals for the encapsulation of repellent active ingredients and/or excipients. Natural sourced polymeric species (e.g., algin, chitosan) may be preferred over synthetic species.

The compositions of the present invention may be filled and stored into drums, bottles, and sachets. The drums may range in volume from approximately 1 to 55 gallons or 1 to 200 kg industrial drums. The bottles may range in volume from approximately 30 to 1,000 ml. The sachets (or sealed pouches), intended as unit dosage forms, may range in volume from approximately 1 to 20 ml.

Multi-dosage forms, such as kits of multiple unit dosage forms, containing 2 to 60 dosage forms, for example containing 7, 14, 30, or 60 unit dosage forms, may be provided. Preferably, the multi-dosage forms contain sufficient unit dosage forms for administration over a period of 7 or 30 days. The unit dosage forms may be heat-sealed sachets formed with synthetic or metallic-synthetic films. The sachets may also contain a synthetic or natural cloth intended as a convenient repellent wipe on skin surfaces.

Compositions of the present invention are essentially encapsulated emulsions in the form of lotions and/or creams. Furthermore, the compositions may consist of aqueous and/or non-aqueous suspensions or dilutions of lotions and/or creams, wherein the dilutions are prepared prior to application to the skin, hair, or clothing. For instance, a lotion composition of the present invention with 10-20 percent active ingredient, could be diluted 1:5, 1:10, 1:50, or 1:100 in water or another solution or solvent (e.g., ethanol or ethanol:water). The diluted repellent solution could be applied immediately on a human, or non-human mammal (e.g., horse, cow, dog, cat), or clothing.

A human or non-human mammal (e.g., horse, cow, dog, cat) may benefit from topical application immediately or within minutes, and the duration of effective repellent activity may last for 6, 8, 10, 12, or 14 hours, depending on the species of insect or non-insect arthropod pest, and wherein the insects are one or more species of mosquito, fly, sandfly, black fly, louse, chigger, bed bug, a vector of trypanosomiasis, bee, Halictid, wasp, and hornet, and the non-insect arthropod pests are one or more species of tick, chigger (Trombiculidae), spider, and scorpion.

The complete protection time (CPT) is the number of minutes (or hours) elapsed between topical application and the first landing and/or probing (biting) of the insect. Confirmed first bite (CFB) is the second incidence of an insect probing (biting).

A variety of insect-borne and non-insect arthropod pest-borne diseases, and the symptoms thereof, may be prevented by treatment of skin, hair, or clothing with an effective composition or method of the present invention. The diseases include, but are not necessarily limited to, one or more of malaria, dengue fever, Lyme disease (Borreliosis), Leishmaniasis, Chagas disease (trypanosomiasis), Zika fever, equine encephalitis, and yellow fever.

### Examples

### Example 1) Compositions with IR3535®

A prototype repellent formulation was developed based in part upon encapsulation methods known in the art, for example, the methods taught in U.S. Pat. No. 8,039,015. U.S. Pat. No. 8,039,015 discloses methods of encapsulation of an immiscible (hydrophobic) inner core material containing the active ingredient with an outer wall created by the chemical bonding of a Lewis acid (e.g., carboxymethylcellulose) and a Lewis base (e.g., benzalkonium chloride). U.S. Pat. No. 8,039,015 specifically discloses methods of producing sodium carboxymethylcellulose plus benzalkonium chloride encapsulated DEET formulations within Examples 1-6. However, unlike the disclosure of U.S. Pat. No. 8,039,015 which contained DEET as an active ingredient, the new formulations of the present invention contain the repellent active ingredient IR3535®, selected because it lacks an odor and it does not produce an oily feel on skin. In addition, the present invention diverges substantially from the Examples of U.S. Pat. No. 8,039,015 with regard to the selected excipients, including the Lewis acid(s) and Lewis base(s).

The following essential categories of excipients were desired for formulation development for an insect or non-insect arthropod pest repellent lotion product: IR3535® as the active ingredient, emollient, emulsifier, encapsulation polymer, thickener, film former, preservative, and water, with the water present at 60-80 percent, and more preferably at approximately 70 percent. Two optional categories of excipients included citric acid as an antioxidant and/or pH reducer, and fragrance. The “Generic Ingredients List” for IR3535®-containing formulations, which was utilized to conceive of a formulation prototype is referred to as prototype version 1.0. (Table 1).

TABLE 1
Generic Ingredients List to Generate Prototype Microencapsulated
Emulsion Formulation Version 1.0

| Category | Chemical Name or Class | % (w/w) |
|---|---|---|
| Active | ethyl butylacetylaminopropionate | 20 |
| Emollient | acyclic alkane | ~2 |
| Emulsifier | sorbitan fatty acid ester | ~1 |
| ″ | polysorbate | <1 |
| Encapsulation | algin + cellulose | <1 |
| ″ | chitosan | <1 |
| ″ | xantham gum | <1 |
| Thickener | polyamide | ~1 |
| ″ | cetyl alcohol | <1 |
| ″ | carrageenan | <1 |
| Film former | silicone | ~1 |
| ″ | non-silicone | ~1 |
| Antioxidant/pH | citric acid (optional) | <1 |
| Preservatives | preservatives | <1 |
| Fragrance | fragrance (optional) | <1 |
| | active + excipients | ~30 |
| | water | ~70 |

When these formulations, including prototype version 1.0, all containing IR3535® (e.g., at 20% w/w) were subjected to stability testing at ambient room temperature (ca. 20°-25° C.) and accelerated temperatures (e.g., 40° C. and/or 50° C.), the microencapsulated emulsions unexpectedly manifested rapid reductions from the initial pH over the course of weeks to months. The repellent lotion prototype version 1.0 unexpectedly manifested rapid reductions in pH by 1.0 unit within 5.1 weeks at 40° C. and 16.4 weeks at ambient room temperature (20°-25° C.). A variance of pH of 1.0 unit or greater within a predetermined time (e.g., 12 months at ambient room temperature) was deemed as an undesirable property; it may be used as a determinant of the imputed or formal shelf life of a product.

The pH drift underscores that some component(s) of the formulations, including those within prototype version 1.0, were undergoing chemical hydrolysis or degradation, as evidenced by increased concentration of hydronium ions (H₃O+) or protons, detected by a pH meter and/or pH test strips. Lower pH and/or the presence of degradants within the formulations are considered to be disadvantageous to human or animal skin. Note that none of these formulation variants, including version 1.0, contained trisodium citrate, even though some formulation variants contained citric acid (optional) as an antioxidant/acidifier. It may not be predicted a priori which formulation components and combinations may be suitable to provide chemical and physical stability for insect or non-insect arthropod pest repellent compositions, and particularly for encapsulated emulsions of IR3535®. Thus, empirical studies of chemical and physical stability parameters must be performed in the laboratory.

Achieving an optimal pH during formulation of encapsulated emulsions as well as in a final commercial product can be challenging. For instance, the demands of the formulation development needed to accommodate at least three substantial variables: (1) According to the manufacturer of IR3535® (ethyl butylacetylaminopropionate; Merck KGaA) the ideal pH for IR3535® is between 6.0-6.5. A challenge of using IR3535® as the selected active ingredient in these encapsulated repellent emulsion formulations, is that to adequately solubilize, suspend, or emulsify the IR3535® and the other essential excipients, a pH lower than its inherent ideal pH of IR3535® can be desirable; (2) Chitosan is a sugar polymer that is depolymerized in acidic conditions, but forms polymers when neutralized; and (3) For human skin a pH of approximately 5.0-6.5 is appropriate, and approximately 5.5 is often preferable or optimal in topical consumer products. These three parameters are in apparent opposition; logic suggested that a low pH might be desirable in the process of producing an IR3535®-containing encapsulated formulation, but higher pH (ca. 5.0-6.5) is desirable to maintain the active ingredient's stability and for application of the formulation to human skin to avoid unnecessary skin irritation.

It follows that the manufacturing process steps (and sequence order) in preparing the formulations may also impact ingredient solubilization, suspension, emulsification, and encapsulation in a pH-dependent manner. Thus, empirical formulation development and analyses via a reiterative process can refine from an initial prototype formulation toward preferred compositions of the present invention.

In multiple efforts to ameliorate the undesirable reductions in pH and concentration of the active ingredient, attempts were made to chemically stabilize the formulation(s), including the following examples: (a) addition of a strong base, 0.33% Sodium Hydroxide (NaOH), to increase the initial pH during manufacturing; (b) addition of a strong base, 1.51% or 3.84% NaOH, to restore (increase) the pH after the formulation had manifested substantial pH loss, although this was attempted as a post-manufacturing palliative approach that is not feasible for use during manufacturing; (c) addition of Citric Acid+NaOH in a titration ranging from 0.38% Citric Acid+0.22% NaOH to 3.0% Citric Acid+2.19% NaOH; (d) addition of Citric Acid+Trisodium Phosphate in a titration ranging from 0.22% Citric Acid+0.58% Trisodium Phosphate to 0.82% Citric Acid+2.17% Trisodium Phosphate; (e) addition of an antioxidant, butylated hydroxytoluene (BHT), at 0.1% or 1.0%; and (f) addition of an antioxidant, BHT, at 0.1% or 1.0% in combination with a relatively low level of Trisodium Citrate/Citric Acid buffer (0.45% Trisodium Citrate+0.032% Citric Acid), with the intent of sufficiently retarding the chemical instability.

None of these multiple empirical approaches adequately addressed the chemical instability issues. The results demonstrate that it cannot be predicted a priori which excipients may be capable of improving pH stability characteristics of the encapsulated emulsion compositions. Furthermore, the results demonstrate that it cannot be predicted as to which excipients may be capable of improving chemical stability characteristics of the active agent, IR3535® The chemical instability persisted even when a strong base (NaOH) or an antioxidant (BHT) was included. In addition, one of ordinary skill in the art of formulation science would understand that the back-neutralization of a Citric Acid solution with Sodium Hydroxide is inherently equivalent to the use of a Sodium Citrate (or hydrates thereof) plus Citric Acid buffer solution, differing only in order of addition of the component parts.

In aggregate at least six separate empirical design alterations were tested within the framework of the Version 1.0 formulation template: (1) NaOH as a base; (2) NaOH as a base and added post-manufacturing after the formulation evidenced pH loss; (3) NaOH as a base and Citric Acid as an acid; (4) Trisodium Phosphate as a base and Citric Acid as an acid; (5) BHT as an antioxidant; and (6) BHT as an antioxidant in combination with a relatively low level of Trisodium Citrate/Citric Acid buffer. Nevertheless, none of these empirical approaches resolved the issue of the pH of the formulation lowering rapidly over time.

These findings (above) further underscored the problems and confounding variables to be addressed as the basis of the present invention. In summary, the problematic issues faced during formulation development included: (a) most notably the unexpected rapid pH loss; (b) undesirable loss of IR3535®; (c) an a priori intuitive preference for a lower pH for solubility, suspension, emulsification, and encapsulation during formulation development; (d) whereas, human skin prefers a higher pH of ca. 5.0-6.5, and more preferably 5.5; and (e) attempts to maintain appropriate pH properties by the addition of NaOH or an antioxidants (e.g., BHT) were not sufficient. Thus, multiple attributes of the excipient components, concentrations, and sequence (order) were evaluated empirically for the capability to retard the chemical instability in topical compositions for insect or non-insect arthropod pest repellent compositions.

Therefore, to prevent the rapid pH reduction and unacceptable loss of the active ingredient, multiple formulations were prepared that contained Trisodium Citrate in addition to Citric Acid (referred to as version 2.0) with an initial pH of 6.2, an example of which is demonstrated in the following Table 2:

TABLE 2
Ingredients List for Microencapsulated Emulsion
Composition Version 2.0 (initial pH 6.2)

| Category | INCI name | % (w/w) |
|---|---|---|
| Active | ethyl butylacetylaminopropionate | 20.000 |
| Emollient | isoeicosane | 2.000 |
| Emulsifier | sorbitan oleate | 1.000 |
| ″ | polysorbate 20 | 0.110 |
| Encapsulation | algin + cellulose | 0.350 |
| ″ | chitosan | 0.007 |
| ″ | xantham gum | 0.347 |
| Thickener | polyamide-3 | 1.000 |
| ″ | cetyl alcohol | 0.500 |
| ″ | carrageenan | 0.347 |
| Film former | dimethicone | 1.000 |
| ″ | acrylates copolymer | 1.000 |
| pH buffer | trisodium citrate, dihydrate | 2.024 |
| ″ | citric acid | 0.095 |
| Preservative | Phenoxyethanol + | |
| ″ | ethylhexylglycerin | 0.700 |
| Fragrance | admixture | 0.150 |
| | active + excipients | 30.630 |
| | water | 69.370 |

The fragrance is optional.

Formulations with two chemical versions of Sodium Citrate were studied, in the dihydrate form and the monohydrate form. The molecular weights of each vary slightly. Thus, to maintain equimolar Citrate concentrations, less of the monohydrate is required relative to the dihydrate. The dihydrate form is routinely used in commercial consumer products and foods, is less expensive, and may be preferable for these reasons.

Formulation variants were also prepared with a concentration range of Sodium Citrate:Citric Acid to identify critical amounts of the buffer. Concentrations ranged from 0.2 to 5.0 percent. In general, the concentration which provided the desired chemical stability and physical aspects was approximately 2 percent, although noting that 1 percent provided some benefit. As another example of a formulation variant of the present invention, composition version 2.55 is identical to version 2.0 (Table 2, above), except it contains 1.80% Trisodium Citrate Dihydrate: 0.31% Citric Acid at initial pH 5.5, and 69.38% water. This initial pH of 5.5 units may be preferable for human skin.

### Example 2) Method of Preparation of Microencapsulated Emulsion Repellent Composition Version 2.0

1. In a heated mixing container, combine the emollient, the emulsifiers, two of the thickeners (polyamide-3, cetyl alcohol), and the film former dimethicone (Phase A). Commence heat to 90° C. Stir until melted Phase A is completely homogenous.
2. Discontinue heating, and when temperature is below 80° C., add IR3535® (Phase A1) gradually, stirring to incorporate.
3. While Phase A (including Phase A1) is cooling, in a separate heated container prepare Phase C: add citric acid into the purified water and stir until completely solubilized, then add the trisodium citrate (dihydrate). Once the trisodium citrate is completely solubilized the pH should be approximately 6.2. Add the final amount of purified water to complete the final weight of Phase C and begin heating to 50° C.
4. In a separate container mix the (dry) encapsulation ingredients and the thickener carrageenan (Phase B), then add Phase B into Phase A slowly and gradually, stirring to avoid local freeze-out of gel. Mix thoroughly.
5. When Phase C is at 50° C., add phase NB into phase C. When all Phase A/B has been added to Phase C, re-commence stirring. Shear hard and homogenize. Continue to shear with the homogenizer to form creamy white emulsion.
6. Add the film former acrylates copolymer (Phase D) and the preservatives (Phase E) into the emulsion sequentially with stirring, while cooling the product.
7. If required, add additional purified water for appropriate final weight.
8. Check pH of the final emulsion is between 6.0 and 6.5. For composition version 2.0 the target pH is 6.2.
9. Add (optional) fragrance (Phase F) into the emulsion once the temperature is below 40° C., Mix thoroughly.

### Example 3) pH Stability and Chemical Stability Assessments of Compositions: Estimated (Imputed) Shelf Lives Via Linear Regression Analyses

The primary goal was to limit the reduction in pH to no more than 1.0 unit within 12 months (and preferably longer than 12 months) at ambient room temperature (RT), to avoid chemical instability of the excipients and active ingredient. This increased pH stability is also expected to stabilize the concentration of IR3535®, so that it does not exceed 5-6% loss over the imputed or formal shelf life. The slopes of the linear regressions (with r2 values>0.9) were extrapolated into imputed (estimated) weeks to reach a pH loss of 1.0 unit (e.g., from an initial pH of 6.2 downward to 5.2 for version 2.0, or from an initial pH of 5.5 downward to 4.5 for version 2.55).

TABLE 3
Elapsed Time (Weeks) to Reach a pH Reduction of 1.0 Unit

| | 1.0 | 2.0 | 2.0 | 2.55 |
|---|---|---|---|---|
| Version | — | (Bd2) | (Bi2) | (20a) |
| Buffered pH | na | 6.2 | 6.2 | 5.5 |
| Weeks @ 40° C. | 5.1 | 27.2 | 27.8 | 90.1 |
| Weeks @ RT | 16.4 | 94.3 | 68.5 | 384.6 |

Version 1.0 is unbuffered; Version 2.0 (Bd2) has ˜2% Sodium Citrate: Citric Acid buffer with Sodium Citrate monohydrate; Version 2.0 (Bi2) is ˜2% Sodium Citrate:Citric Acid buffer with Sodium Citrate dihydrate, same as Table 2 composition; Version 2.55 (20a) has a modified ratio of ˜2% Sodium Citrate:Citric Acid buffer and using Sodium Citrate dihydrate.

Table 3 reveals that linear regression analyses of weekly measurements of the unbuffered repellent lotion prototype version 1.0 manifested rapid reductions in pH by 1.0 unit within 5.1 weeks at 40° C. and 16.4 weeks at ambient room temperature (20-25° C.). These times were deemed to be undesirable for a commercial product. At 40° C. the linear regression of the slope revealed that the buffered version 2.0 (two variants; Bd2 and Bi2) at initial pH of 6.2 reduced the rate of pH drift by 5.3× and 5.5×, respectively. At 40C the buffered version 2.55 (20a) reduced the rate of pH drift by 17.7×. At ambient room temperature (20°-25° C.) the buffered versions 2.0 (Bd2 and Bi2) reduced the rate of pH drift by 5.8× and 4.2×, respectively. And, at ambient room temperature the buffered version 2.55 (20a) reduced the rate of pH drift by 23.5×.

If the time to arrive at a loss of 1.0 pH unit is selected as one of the undesirable criteria for establishing the imputed or formal shelf life, then the Sodium Citrate:Citric Acid buffered versions 2.0 (Bd2 and Bi2) can last for at least one year prior to a reduction in pH of 1.0 unit. Furthermore, version 2.55 (20a) with an initial pH of 5.5 is expected to be stable for multiple years. Thus, an initial formulation pH of 5.5 (or approximately 5.5) is a preferred embodiment. These results indicate that chemical stability, of which pH is an important marker, has been substantially enhanced by the compositions of the present invention, and especially so at an initial buffered pH of 5.5.

The shelf-life estimates may also be established as a function of the concentration of the active ingredient (e.g., IR3535®), but the variance should not exceed 5 or 6% loss of the active ingredient over the duration of the imputed or formal shelf life. The concentration of IR3535® of version 2.0 was assessed at 26 weeks at 40° C. and was determined to conform to the acceptable variance. Thus, the Sodium Citrate—Citric Acid buffered formulation 2.0 (initial pH of 6.2) stabilized not only the pH but also the active ingredient from hydrolysis/degradation. As previously noted, the addition of Sodium Hydroxide to a Citric Acid-containing formulation did not stabilize the pH. The compositions formed by inclusion of Trisodium Citrate (or hydrates thereof) was surprisingly effective in stabilizing the pH and protecting and/or stabilizing the active ingredient against hydrolysis/degradation.

The physical properties of Sodium Citrate—Citric Acid buffered formulations versions 2.0 and 2.55 were assessed at the time they were prepared and at various timepoints thereafter at ambient room temperature (20°-25° C.) and “accelerated” temperature (40° C.). The formulations were stable at ambient and accelerated temperatures with regard to color, texture, and viscosity. Thus, the Sodium Citrate—Citric Acid buffering did not compromise the physical properties of these formulation versions. They did not undergo phase separation or flocculation.

### Example 4) Efficacy of Compositions on Humans Against Insects in Laboratory Studies

The repellent efficacy against mosquitos was assessed for buffered version 2.0 and the unbuffered prototype version 1.0. A laboratory efficacy test (arm-in-cage) was conducted for the version 2.0 on 10 human participants against the mosquito species Aedes aegypti at Laboratorios Ecolyzer LTDA. (Sao Paulo, Brazil). The dose was 1 g of repellent lotion per 600 cm² applied to the forearm. Sufficient insect biting pressure was assured at each time point. None of the participants experienced a confirmed first bite (CFB; i.e., second bite) prior to 13 hours. At 13 hours into the study 4 of the 10 participants experienced mosquitos landing and/or biting, thus the Complete Protection Time (CPT) for the protection against the mosquito species Aedes aegypti was 13 hours (based upon a CFB criterion). The prototype version 1.0 manifested a CPT of 12 hours using the same experimental method and facility. Thus, both compositions (versions 1.0 and 2.0) displayed long duration efficacy, which was superior to other commercial products using IR3535® as an active ingredient. As a direct and relevant comparison conducted within the same laboratory, the commercial “Jungle Formula for Kids” containing 20% IR3535® provided a CFB of only 4 hours (and an average protection time of only 7 hours). Furthermore, the inventors of the present invention are unaware of any commercial repellent products containing IR3535® that have been approved by a governmental regulatory authority as effective for up to 12 hours against mosquitos, although some products are approved for lower duration efficacy. The 13 hour CPT for version 2.0 is surprisingly superior to the efficacy of numerous commercial products containing DEET or IR3535®.

### Example 5) Treatment of Humans with Repellent Compositions

The lotion versions 2.0 and 2.55 are applied to the skin of humans for the in-life protection against bites from mosquitos and/or flies. The composition versions 2.0 and 2.55 are cosmetically acceptable, quickly dry on the skin, do not have an oily or greasy feel after drying on the skin, and do not have an unpleasant aroma. The formulation may be applied pro re nata (prn; “as needed”) on occasion and/or once or multiple times daily. Humans may apply the compositions during the daytime and at night to skin to protect from mosquito and/or flies.

### Example 6) Efficacy of Repellent Compositions on Humans Against Insects In-Life

Human (or nonhuman mammals) can be exposed in-life to mosquitos, flies, and/or sand flies in outdoors environments known to contain the relevant species of biting insects. A common test paradigm is to treat a group of subjects' exposed skin surfaces with the repellent composition, and the elapsed time is recorded in 30 or 60 minute intervals until the confirmed first bite (CFB) occurs, i.e., when the first individual within a group of subjects experiences a CFB (at the time of the second bite). This represents a minimum limit; some individuals may or are likely to experience protection beyond the CFB threshold.

A study design resembling this type has been performed with the unbuffered prototype version 1.0 in Florida (US) and yielded an average protection time of 14 hours against mosquitos. Note that an average protection time can or will often be a higher number in hours than a CFB threshold. It is anticipated that the versions 2.0 and 2.55 will display similar in-life efficacy to that of version 1.0, as the unbuffered 1.0 and buffered 2.0 and 2.55 versions all have encapsulated IR3535® at a nominal concentration of 20 percent.

An alternative test paradigm is to use one exposed limb treated with the repellent composition and the other limb is either untreated or treated with a control (e.g., placebo or other repellent formulation), for intra-individual comparison. However, the aroma generated by the material on the contralateral arm might interfere with the insects' behavior on the arm treated by the test composition.

### Example 7) a Trial to Prevent the Clinical Symptoms of Disease

Primary Objective: To decrease clinical symptoms of an endemic insect-borne disease.

Study materials: A topical lotion composition of the present invention, and a control (e.g., a topical placebo lacking an insect repellent active ingredient or a commercial insect repellent formulation).

Study population: Adults age 19+, male and female, located within an endemic insect-borne disease geographic region, in an outpatient environment.

Subject Groups A & B: Patients within an endemic region can be treated as outpatients. The route of administration of the composition and the control formulation is topical and is applied one or twice daily. Group A is a composition of the present invention and Group B is the placebo control or a commercial insect repellent product containing DEET or IR3535®.

Sample Size: The sample size will be between approximately 20-100 patients in each arm depending on recruitment and accumulation rates, and a priori or interim analyses.

If a single group of patients is treated sequentially in a cross-over design with the composition of the present invention during one period and placebo during another period (i.e., treatment first, placebo second; or placebo first, treatment second), then a relatively low number of subjects is required. It is estimated that 20 enrolled patients (and permitting 3 dropouts) receiving both test articles separately in time is sufficient to provide a 90 percent probability (p-value 0.10) at a 0.80 Standard Deviation (SD) in clinical effect. A prospective statistical power analysis is recommended, but in general the minimum enrollment per treatment arm is approximately 20 subjects. Larger groups would be required if the two groups were independent and/or the clinical effect were smaller than 0.80 SD.

Study Duration: The length of time to enroll and treat a maximum of 200 patients is estimated at 6-12 months, depending on the number of sites. If lower numbers of subjects are deemed adequate, then a shorter recruitment time is sufficient. The course of prophylaxis is estimated to last from approximately 1-6 months per patient, depending on the symptoms to be assessed and the pathogenesis of the disease.

Methodology: A randomized and blinded study design wherein the patient (subject) and study director informing the patient do not know the identity of the compositions of the present invention and control formulations applied to the skin is preferred if feasible. Blinding to the patient might not be possible if the formulations have observable differences in characteristics (e.g., aroma). The symptoms will be those common to the specific insect-borne disease (e.g., cutaneous erythema, inflammation, skin irritation, fever, headaches), and might include diagnostic tests for parasites within the blood, or RNA or DNA of the pathogen within the blood.

Group A: Once or twice daily application of a specific dosage of the composition of the present invention to the skin normally exposed without clothing to the biting insects. For instance, one sachet of approximately 2-5 ml of the composition may be applied to the head and neck region.

Group B: Once or twice daily application of a specific dosage of the placebo control or a commercial insect repellent formulation to the skin normally exposed without clothing to the biting insects. For instance, one sachet of approximately 2-5 ml of the composition may be applied to the head and neck region. The patients (subjects) are 1:1 age and/or sex stratified relative to Group A patient population.

Exclusions: Sensitivity or allergy the composition of the present invention or control formulation, if known. If sensitivity or allergy is revealed during the study, then discontinue use of the composition of the present invention or control formulations.

Primary Endpoint: Reduced disease symptoms for group A vs group B after multiple weeks or months of prophylaxis.

Secondary Endpoints: (1) Reduced days of routine lifestyle disruption in group A vs group B; (2) Each of the study endpoints can be compared to published standard-of-care patient outcomes, either as the control arms of the published studies or as the formulation treatment arms of the published studies. This type of comparison may be especially informative if the number of patients is limited in the control group B, perhaps due to compassionate care or Institutional Review Board considerations limiting the number of placebo-treated patients to minimize risk. Thus, group A vs published standard-of-care groups may serve as an alternative to a direct group A vs group B comparison.

Anticipated Results: Using a crossover design with approximately 20 patients or more per study arm, it is anticipated that 10, 20, 40, 60, or possibly 80 percent of the subjects will experience reduced clinical symptoms relative to the same group administered the placebo. The reduction in symptoms is anticipated within weeks or months of commencing the treatment and is expected to last the duration of the course of treatment.

### Example 8) Efficacy of Compositions on Humans Against Non-Insect Arthropod Pests: Laboratory Study

To establish repellent efficacy against non-insect arthropod pests, an experiment can be conducted with ticks applied to the skin of human subjects in a laboratory. A portion of the skin is treated and an adjacent portion is untreated. The ticks are placed on a portion of the treated skin, and the resident time of the tick (or other non-insect arthropod pest) within that zone is recorded.

For instance, a pilot laboratory study design of this type has been performed with several human subjects using the unbuffered prototype version 1.0 at the Arthropod Control Product Test Centre (ARCTEC), London School of Hygiene & Tropical Medicine; London UK) and yielded an average repellency time of ca. of 15-16 hours against ticks. A similar result with buffered versions 2.0 and 2.55 is anticipated.

### Example 9) Anti-Infective Properties of Repellent Compositions Containing IR3535®

The compositions versions 1.0 and 2.0 of the present invention have rapid-acting anti-infective properties within 60 seconds against bacterial species in vitro—Pseudomonas aeruginosa, Escherichia coli, Staphylococcus aureus, and Enterococcus hirae. High potency (i.e., five log order killing) anti-bacterial effects were observed for three of four strains. This anti-infective effect is perhaps due to IR3535® (14), the preservatives—phenoxyethanol and ethylhexylglycerin, the surfactant/emulsifiers, and/or other excipients. Composition versions 2.0 and 1.0 were close to the effectiveness of a common antiseptic—70% isopropyl alcohol, but slightly less effective. The two sustained-release repellent lotions exhibited 99.9% killing.

Inherent anti-infective properties of a topical repellent formulation may provide coincidental benefits to mammalian skin, for instance in the prophylaxis or treatment of infections in compromised skin (e.g., abrasions, lacerations, insect bites, etc.). Thus, anti-infective properties may be a preferred embodiment of the present invention.

### Example 10) Repellent Compositions with Other Non-DEET Active Ingredients

Compositions of the present invention were prepared that were similar to version 2.0, albeit substituting IR3535® with alternative non-DEET active ingredients, including natural active ingredients—oil of lemon eucalyptus (OLE) or clove leaf oil, at concentrations of 10 and 20% w/w, and 5% w/w respectively. These non-DEET alternatives to IR3535®-containing topical formulations may be preferred in selected commercial and/or regulatory contexts, as the active ingredients are derived from natural sources, rather than synthetic means (i.e., organic chemistry synthesis). The OLE-containing and clove leaf oil-containing compositions chemically stabilized as described in the present invention exhibited no more than 1.0 pH unit variance after 12 weeks at 40° C.

These OLE and clove leaf oil compositions manifest potent odors to humans, thus making them less preferred for application to human skin, hair, or clothing. However, these compositions are useful in the treatment of other mammals, especially those living in outdoor environments, such as horses, cattle, and/or outdoor pets.

### Example 11) Dilution of Composition Prior to Application to Skin, Hair, or Clothing

Compositions of the present invention may be diluted immediately prior to application to skin, hair, or clothing of a human or nonhuman mammal. The diluents may be water, buffered water, alcohol (e.g., ethyl alcohol or isopropyl alcohol), and/or a biocompatible topical lotion. Dilution may be preferable for application to hair, including the hair of pets of livestock in need of protection from biting insects or non-insect arthropod pests. Furthermore, it may be preferable to prepare the dilution immediately prior to application to the human or nonhuman mammal, as the formulation stability properties may change in the more dilute form. For instance, pilot studies of diluted formulations of the present invention have indicated the possibility of phase separation within days of preparation.

### Example 12) Commercial-Stage Long-Duration Efficacious Topical Repellent

In view of the Examples cited above, a commercially-viable repellent lotion product is obtained, which demonstrates the following aggregate features: (a) encapsulation of IR3535® within an emulsion (or microemulsion) using Lewis Acid-Lewis Base adducts; (b) long-term pH stability of the formulation using sodium citrate-citric acid buffer; (c) long-term chemical stability of IR3535® using sodium citrate—citric acid buffer; and (d) long-duration mosquito repellency by IR3535®. Furthermore, buffered formulation versions 2.0 and 2.55 have been produced in kilogram+manufacturing-scale processes, with demonstrated sufficient formulation stability characteristics in laboratory tests.

For comparison to the present invention, PCT International Application Publication No. WO 2020/065541 and US Patent Application Publication No. 2022/0047472 may be characterized as disclosing optically translucent microemulsions which may contain IR3535®, alginic acid, chitosan, and xanthan gum. The disclosures provide no evidence of sodium citrate—citric acid buffer capacity, nor demonstrations of pH stability of compositions and IR3535® stability, nor IR3535® mosquito repellency efficacy within the microemulsion.

The PCT International Application Publication No. WO 2020/065541 and US Patent Application Publication No. 2022/0047472 disclosures are silent with regard to the problems of rapid pH loss and loss of IR3535® in microencapsulated repellent formulations, as revealed in the microencapsulated emulsion compositions of the present invention.

It is demonstrated herein that microencapsulated repellent formulations can exhibit rapid pH loss and loss of stability of IR3535® in a formulation intended for personal use. However, the present invention solves a problem of IR3535® microencapsulation formulation instability by pH buffering the IR3535® formulation with, specifically, sodium citrate—citric acid buffering. The IR3535® emulsion formulation with, specifically, sodium citrate—citric acid unexpectedly provides a chemically and physically stable repellent formulation containing IR3535® as the active ingredient, and provides the required shelf-life stability and long-duration insect repellency desired for a commercial insect or non-insect arthropod pest repellent product.

### REFERENCES

1. Kasting G B, Bhatt V D, Speaker T J. Microencapsulation decreases the skin absorption of N,N-diethyl-m-toluamide (DEET). Toxicology in vitro: an international journal published in association with BIBRA. 2008; 22(2):548-52.
2. Karr J I, Speaker T J, Kasting G B. A novel encapsulation of N,N-diethyl-3-methylbenzamide (DEET) favorably modifies skin absorption while maintaining effective evaporation rates. J Control Release. 2012; 160(3):502-8.
3. Gomes Fernandes M R, Cruz Lopes L, Suguimoto Iwami R, Del Grossi Paglia M, Mateus de Castilho B, Maicon de Oliveira A, et al. Efficacy and safety of repellents marketed in Brazil against bites from Aedes aegypti and Aedes albopictus: A systematic review. Travel Med Infect Dis. 2021; 44:102179.
4. Barnard D R, Bernier U R, Posey K H, Xue R D. Repellency of IR3535, KBR3023, para-menthane-3,8-diol, and deet to black salt marsh mosquitoes (Diptera: Culicidae) in the Everglades National Park. Journal of medical entomology. 2002; 39(6):895-9.
5. Cilek J E, Petersen J L, Hallmon C E. Comparative efficacy of IR3535 and deet as repellents against adult Aedes aegypti and Culex quinquefasciatus. J Am Mosq Control Assoc. 2004; 20(3):299-304.
6. Thavara U, Tawatsin A, Chompoosri J, Suwonkerd W, Chansang U R, Asavadachanukorn P. Laboratory and field evaluations of the insect repellent 3535 (ethyl butylacetylaminopropionate) and deet against mosquito vectors in Thailand. J Am Mosq Control Assoc. 2001; 17(3):190-5.
7. Frances S P, MacKenzie D O, Rowcliffe K L, Corcoran S K. Comparative field evaluation of repellent formulations containing deet and IR3535 against mosquitoes in Queensland, Australia. J Am Mosq Control Assoc. 2009; 25(4):511-3.
8. Licciardi S, Herve J P, Darriet F, Hougard J M, Corbel V. Lethal and behavioural effects of three synthetic repellents (DEET, IR3535 and KBR 3023) on Aedes aegypti mosquitoes in laboratory assays. Medical and veterinary entomology. 2006; 20(3):288-93.
9. Nakayama S K, Graf R, Bohlmann A M, Zurlage J, Epstein H, Ranzani M, et al. Influence of Sex Ratio and Density on the Comparative Repellency of IR3535((R)) Against Caged Aedes, Anopheles, and Culex Mosquitoes. Journal of cosmetic science. 2019; 70(4):197-207.
10. Costantini C, Badolo A, Ilboudo-Sanogo E. Field evaluation of the efficacy and persistence of insect repellents DEET, IR3535, and KBR 3023 against Anopheles gambiae complex and other Afrotropical vector mosquitoes. Transactions of the Royal Society of Tropical Medicine and Hygiene. 2004; 98(11):644-52.
11. Naucke T J, Lorentz S, Grunewald H W. Laboratory testing of the insect repellents IR3535 and DEET against Phlebotomus mascittii and P. duboscqi (Diptera: Psychodidae). Int J Med Microbiol. 2006; 296 Suppl 40:230-2.
12. Reynoso M M N, Seccacini E A, Calcagno J A, Zerba E N, Alzogaray R A. Toxicity, repellency and flushing out in Triatoma infestans (Hemiptera: Reduviidae) exposed to the repellents DEET and IR3535. PeerJ. 2017; 5: e3292.
13. Alzogaray R A. Behavioral and Toxicological Responses of Rhodnius prolixus (Hemiptera: Reduviidae) to the Insect Repellents DEET and IR3535. Journal of medical entomology. 2016; 53(2):387-93.
14. lyigundogdu Z, Kalayci S, Asutay Aft Sahin F. Determination of antimicrobial and antiviral properties of IR3535. Molecular biology reports. 2019; 46(2):1819-24.
15. Carroll S P. Prolonged efficacy of IR3535 repellents against mosquitoes and blacklegged ticks in North America. J Med Entomol. 2008; July; 45(4):706-14.
16. Naucke T J, Kröpke R, Benner G, Schulz J, Wittern K P, Rose A, Kröckel U, Grünewald HW. Field evaluation of the efficacy of proprietary repellent formulations with IR3535 and picaridin against Aedes aegypti. Parasitol Res. 2007; June; 101(1):169-77.

1. An insect or non-insect arthropod pest repellent emulsion composition consisting essentially of ethyl butylacetylaminopropionate and one or more emollients, one or more emulsifiers, one or more Lewis Acids for microencapsulation, one or more Lewis Bases for microencapsulation, one or more viscosity thickeners, one or more film formers, a pH buffer of trisodium citrate, or hydrates thereof, and citric acid, and water.

2. The insect or non-insect arthropod pest repellent emulsion composition of claim 1, wherein the emollient is isoeicosane, wherein the emulsifiers are selected from sorbitan oleate, polysorbate 20, and combinations thereof, wherein the Lewis Acids for microencapsulation are selected from algin, xanthan gum, and combinations thereof, wherein the Lewis Base for microencapsulation is chitosan, wherein the viscosity thickeners are selected from polyamide-3, cetyl alcohol, carrageenan, and combinations thereof, and wherein the film formers are selected from dimethicone, acrylates copolymer, and combinations thereof.

3. The insect or non-insect arthropod pest repellent emulsion composition of claim 1, wherein the ethyl butylacetylaminopropionate is present at a concentration of approximately 2 to 22 (wt) percent.

4. The insect or non-insect arthropod pest repellent emulsion composition of claim 1, wherein the Lewis Acids and Lewis Bases for encapsulation in aggregate do not exceed 1 (wt) percent.

5. The insect or non-insect arthropod pest repellent emulsion composition of claim 1, wherein the initial pH is approximately 5.0 to 6.5, and the pH buffer is approximately 0.5 to 3 (wt) percent.

6. The insect or non-insect arthropod pest repellent emulsion composition of claim 1, wherein the composition is in the form of a microencapsulated emulsion.

7. The insect or non-insect arthropod pest repellent emulsion composition of claim 1, wherein the composition is in the form of a concentrated microencapsulated emulsion, wherein the concentrated microencapsulated emulsion may be diluted with an aqueous solution prior to administration.

8. The insect or non-insect arthropod pest repellent emulsion composition of claim 1, wherein the composition exhibits an imputed or formal shelf life of at least 12 months.

9. A method of treating skin, hair or clothing of a mammal to repel insects or non-insect arthropod pests comprising applying the composition of claim 1 to the skin, hair and/or clothing of the mammal.

10. The method of claim 9, wherein the mammal is a human, horse, cow, dog, cat, or other domesticated mammal.

11. The method of claim 9, wherein the insects are selected from one or more species of mosquito, fly, sandfly, black fly, louse, chigger, bed bug, a vector of trypanosomiasis, bee, Halictid, wasp, and hornet, and wherein the non-insect arthropod pests are selected from one or more species of tick, chigger (Trombiculidae), spider, and scorpion.

12. The method of claim 9, wherein a single application of the insect or non-insect arthropod pest repellent composition to the skin, hair, or clothing repels insects of one or more species for a minimum of 10 hours.

13. The method of claim 9, wherein the application of an insect or non-insect arthropod pest repellent composition further prevents symptoms of insect-borne diseases, and wherein the diseases include one or more of malaria, dengue fever, Lyme disease (Borreliosis), Leishmaniasis, Chagas disease (trypanosomiasis), Zika fever, equine encephalitis, and yellow fever.
